(19) United States
(12) Patent Application Publication
(10) Pub. No.: US 2020/0107950 A1
(43) Pub. Date: Apr. 9, 2020

(54) HANDLE FOR DELIVERING A STENT AND DEVICE COMPRISING THE HANDLE

(71) Applicant: HEXACATH, Rueil-Malmaison (FR)

(72) Inventors: Gilles Charles Franklin ASCHER, Neuilly-sur-Seine (FR); Philippe ANDREANI, Paris (FR)

(21) Appl. No.: 16/621,146

(22) PCT Filed: Jun. 25, 2018
(86) PCT No.: PCT/FR2018/051539

§ 371 Date: Dec. 10, 2019

(30) Foreign Application Priority Data
Jun. 27, 2017 (FR) 1755883

(51) Int. Cl. A61F 2/966

(52) U.S. Cl. CPC A61F 2/966; A61F 2002/9517

(57) ABSTRACT

The invention relates to a handle (10) for delivering a stent (2) by retracting a sheath (4) for protecting the stent (2), and is characterized in that it includes a tackle system (20) used to pull the protective sheath (4) over a predetermined retraction distance (D) to uncover the stent and enable the stent to be delivered and expanded. The tackle system greatly reduces the tractive force required to release the stent.

The invention relates to a handle for delivering a stent and a device for delivering a stent comprising the handle. The invention also relates to a method for delivering the stent with said handle.

### PRIOR ART

A device for delivering a stent including a handle 212 that includes a housing 214 and a spool 222, a pushrod 204 with a proximal end 204P connected to the housing and a distal end near to the stent, in which the handle has a linear length Y that is shorter than the linear length X of the stent, is known from document EP 1834610. A sheath 206 constraining the stent at the distal end thereof and having a proximal end 206P is connected to a distal end of a retraction wire 224 connected via the proximal end thereof to said spool 222 is provided. Thus, the rotation of the spool 222 winds the retraction wire, which retracts the constriction sheath 206 of the stent to release the stent.

However, this device has the drawback of requiring the application of a significant retraction force on the spool to retract the constriction sheath of the stent.

### PURPOSES OF THE INVENTION

The main purpose of the invention is to provide a solution that resolves the technical problem of reducing the tractive force that needs to be exerted on the spool to retract the sheath and release the stent at the distal end.

Another main purpose of the invention is to provide a solution that resolves the technical problem of reducing the tractive force that needs to be exerted on the spool according to a solution that reduces this tractive force.

Another main purpose of the invention is to provide a solution that resolves the technical problem of reducing the tractive force that needs to be exerted on the spool that is simple to implement without increasing manufacturing costs and that is technically and medically safe and reliable, enabling industrial-scale manufacture.

### SUMMARY OF THE INVENTION

The invention provides a handle for delivering a stent that is arranged on a catheter by retracting a protective intermediate sheath or tubular element of the stent, including a distal end and a proximal end, the stent is arranged in a constrained manner inside the distal end of the intermediate tubular element or sheath including a tackle system used to pull the protective intermediate sheath or tubular element of the stent over a predetermined retraction distance in order to uncover the stent and enable the stent to be delivered and expanded, characterized in that the tackle system includes a loop on the upstream side that is rigidly connected to the intermediate protective sheath or tubular element, said loop being traversed by a wire performing a tackle function, one end of which is fastened to the housing of the handle, and is therefore static, and the other end is rigidly connected to a control wheel that is mounted in rotation on the housing of the handle.

According to another specific variant embodiment, the handle has a front portion on the side of the stent and a rear portion used to insert the catheter on which the stent is arranged. In a starting position, the tackle system is positioned towards the front of the handle so that, when retracted by traction, the retraction distance is substantially similar to the length of the handle.

According to another specific variant embodiment of the invention, the wheel has a bearing or lateral shaft on which the traction wire is wound.

According to another specific variant embodiment of the invention, the wheel and the related lateral shaft or bearing are in an intermediate position between the front and rear of the handle, the size of the rear portion of the handle being such as to enable the handle to be held comfortably in an operator's hand. In this intermediate position, the operator can hold the handle firmly while turning the wheel, which is large enough that the outer circumferential edge thereof projects outside the handle.

According to another specific variant embodiment, the outer circumferential edge of the handle is notched to facilitate the rotational movement imparted by the operator's finger or thumb when the wheel is arranged on the top of the handle.

According to another specific variant embodiment, a support and change-of-direction shaft for the traction wire coming from the front portion of the handle through the loop is provided inside the rear portion of the handle, which also helps to lower the tractive force that needs to be exerted on the sheath to pull said sheath inside the handle. Thus, the traction wire is fastened at one end to a base rigidly connected to the housing towards the rear portion of the handle, then heads towards the front of the handle to pass through the loop rigidly connected to the protective sheath of the stent, returns to the back of the handle to pass about the support and change-of-direction shaft, and goes back to the front where the second end thereof is fastened to the wheel. Thus, according to this embodiment, the total length of the traction wire is greater than twice the length of the handle and less than three times the length of the handle.

The support and change-of-direction shaft arranged at the rear of the handle can be made of a material that facilitates sliding, such as metal or plastic.

According to another variant, the handle has a reinforcement element at the front to stiffen same and to connect the handle to the catheter device including the stent.

According to another specific variant embodiment of the invention, an element for temporarily blocking a movement of the tackle system is provided. This blocking element can be provided at the wheel to prevent rotation thereof, and in particular at the loop rigidly connected to the intermediate sheath or tubular element, by insertion beneath the loop, for example in the form of a lug of cylindrical or tapered section, in order to raise and compress the loop to block the loop in position until the handle is used.

According to a second aspect, the invention also covers a system for delivering a stent that includes the handle described above.

According to a specific variant embodiment of the invention, this system for delivering the stent includes the handle and a catheter device that is shaped to be inserted into a channel of the human body, in particular a blood vessel, vein, artery and notably a coronary, including an intermediate tubular element or sheath with a proximal end and a distal end; a stent arranged in a constrained manner inside the distal end of the intermediate tubular element; an inner tubular element or sheath with a proximal end and a distal end that is arranged inside the intermediate tubular element and that is long enough that the distal end thereof is positioned at the rear of the stent and blocks the stent in position to prevent the stent from being retracted; the proximal end of the inner tubular element is rigidly connected to the handle in a static position.

According to another specific variant embodiment of the invention, an outer sheath or tubular element with a proximal end and a distal end is positioned outside the intermediate tubular element and is shorter than is the intermediate tubular element so that the distal end thereof is positioned at a predetermined distance upstream of the stent, and the proximal end of the outer tubular element is rigidly connected to the handle also in a static position.

According to another variant embodiment of the invention, the distal end of the intermediate tubular element has a diameter D1 that is greater over the length along which the stent is received to facilitate the insertion thereof, then has an inclined joint resulting in a lesser diameter D2 over the upstream portion of the intermediate tubular element. This diameter D1 substantially matches the diameter D3 of the outer tubular element such as to be positioned against the distal end of the outer tubular element, which is static and forms a stop preventing retraction of the intermediate tubular element.

The person skilled in the art understands that the intermediate tubular element is designed to slide longitudinally between the inner tubular element and the outer tubular element, which remain in a static position relative to the handle. The stent remains in place because the distal end of the inner tubular element remains static and prevents any retraction movement of the stent. The invention therefore enables the stent to be delivered to a precise position.

According to a variant embodiment, the predetermined distance upstream of the stent of the distal end of the outer tubular element or sheath can be substantially equal to the retraction distance of the intermediate tubular element, this retraction distance being at least the length of the stent to be delivered.

Other purposes, features and advantages of the invention are set out clearly in the description below with reference to the attached figures, which show a currently preferred embodiment of the invention, given simply by way of example and without limiting the scope of the invention. The drawings are an integral part of the invention and thus complement the description.

### IN THE DRAWINGS

FIG. 1 is an overview of the system 1 for delivering a stent 2 including a catheter device 100 with the stent 2 in the distal portion, protected inside a protective sheath or tubular element 120 before the stent is released, and a handle 10,

FIG. 2 is a highly magnified cross-section view of the distal portion of the catheter device clearly showing the stent 2 and the inner and intermediate sheaths or tubular elements 110, 120 respectively accommodating the stent 2, and the outer sheath or tubular element 130,

FIG. 3 is an overview of the handle in the open position with the right-hand shell shown in FIG. 1 removed, the handle being made of two matching longitudinal halves or parts or shells that click together. FIG. 3 also shows the left-hand shell 12 to show the inside of the handle, which includes the tackle system according to the invention and the related wheel. FIG. 3 also shows the reinforcement element 18 in place with a tapered front portion arranged outside the handle and an inverted-T-shaped rear portion in which the base of the “T” is seated in a transverse seat and held in the closed position of the handle. FIG. 3 also shows the tackle blocking member 20, for example a pin and including an orifice 91 designed to facilitate retraction of same, ending in two pegs 92 and 93, the front pin 92 in this case passing beneath the loop 30 and the other pin is 93 entering the space between two teeth 52 of the wheel 50,

FIG. 4 is a highly magnified view of the front portion of the handle, centered on the tackle system 20 and with the reinforcement removed for the sake of visibility. The figure shows that the tackle system includes a loop and a traction wire and the two legs of the loop are bonded to the intermediate tubular element or sheath designed to accommodate and protect the stent,

FIG. 5 is a further magnified view of the blocking member for the tackle 20 in FIG. 3,

FIG. 6 is also a highly magnified view of the rear portion of the handle, showing the base 80 for fastening the upper portion of the traction wire 40 on the housing, or in this case the shell 12, of the handle, as well as the support and change-of-direction shaft of the traction wire toward the wheel. This support and change-of-direction shaft is an important component in the tackle system according to the invention that reduces the tractive force,

FIG. 7 is a magnified view of the traction wheel 50 showing the lateral shaft 60 onto which the traction wire 40 is wound, and

FIG. 8 is an overview of the handle in the open position, similar to the view in FIG. 3 but with the loop arranged at the end of traction position to the rear of the handle, which is the release position of the stent, as shown in the magnified portion in FIG. 8.

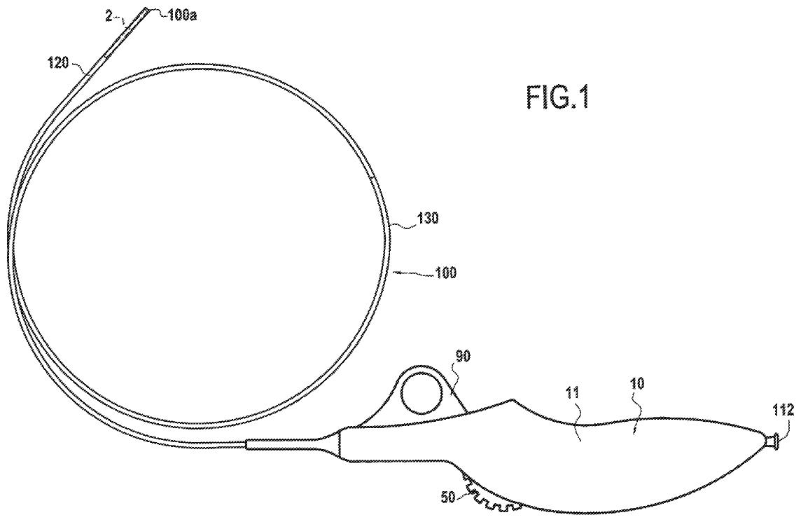

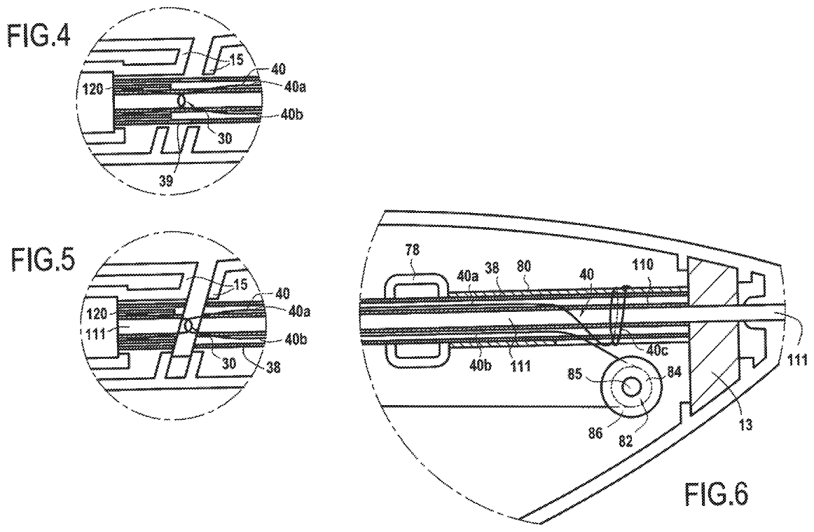

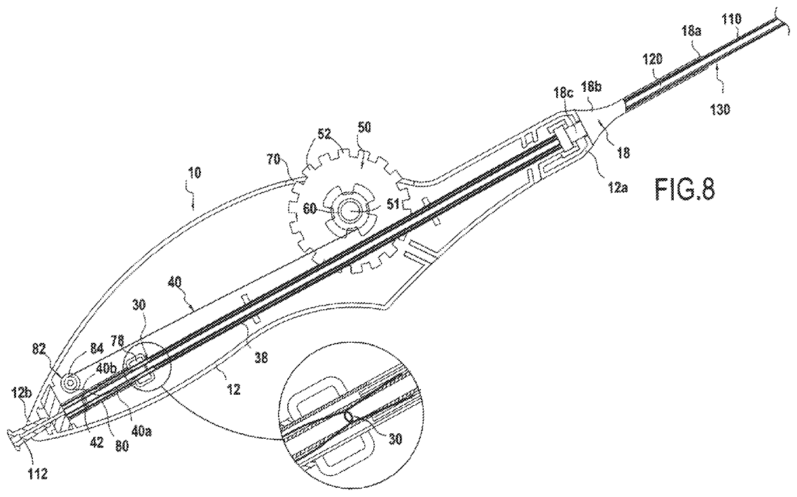

According to a first aspect, and with reference to FIGS. 1 to 8, the invention relates to a new concept for a handle (10) for delivering a stent (2) by retracting a tubular element or sheath (120) protecting the stent (2), in which this element or sheath can be intermediate according to the currently preferred embodiment and be part of a catheter (100), which is is described below. A tubular element (110) for blocking or stopping the stent (2), referred to as “push tubing” or “stop tubing” is also provided, and an outer tubular element or outer sheath (130) designed to facilitate insertion into a channel such as a blood vessel is also advantageously provided. In the remainder of the description, the terms “tubular element” and “sheath” are used without distinction.

This currently preferred embodiment also relates to a system for delivering a stent (2) that includes this handle (10) that constitutes the essence of the invention.

The stent delivery system usually includes a catheter device (100) designed to enable a predetermined retraction distance D determined in part by the length of the handle (10) in order to uncover the stent (2) and to enable the stent to be delivered and expanded. It can be easily understood that the predetermined retraction distance D is also a function of the length of the stent (2) to be released. The lengths of the stent (2) to be released are variable and can reach 150 mm or even 200 mm. It can be easily understood that a range of handle lengths can be provided to adapt to the length of the stent (2).

According to the invention, the handle is characterized in that it includes a tackle system (20) used to pull the protective intermediate sheath (120) of the stent (2) over a predetermined retraction distance (D) in order to uncover the stent and enable the stent to be delivered and expanded.

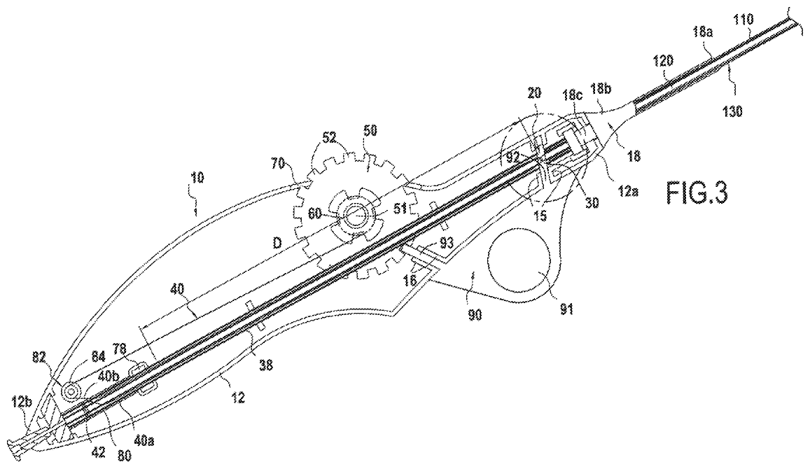

According to a specific variant embodiment, the handle is characterized in that the tackle system (20) includes a loop (30) that is rigidly connected to the intermediate protective sheath (120) on the upstream side, and the two legs of the loop are rigidly connected to the protective sheath (120). This loop (30) and the rigid connection thereof to the intermediate sheath (120) are clearly visible in magnified FIGS. 3, 4 and 5. The loop (30) is traversed by a traction wire (40) that performs a tackle function, one end (40a) of said wire being fastened statically to the housing or shell, in this case the left-hand shell (12), of the handle (10) and the other end (40b) being rigidly connected to a control wheel (50) that is mounted in rotation on the housing or shell (12) of the handle.

According to another specific variant embodiment, the handle (10) has a front portion (12a), in this case on the shell (12) on the side of the stent (2), and a rear portion (12b) that is provided with a cannula (112) used to insert a guide wire (not shown) passing through the central channel (111). In the starting position shown in FIGS. 1 to 8, the tackle system (20) is positioned towards the front (12a) of the handle (10) so that, when retracted by traction, the retraction distance (D) is substantially close to the length of the handle, this length being in turn calculated as a function of the length of the stent (2).

According to another specific variant embodiment, the wheel (50) has a lateral shaft (60) on which the traction wire (40) is wound.

According to another specific variant embodiment, the wheel (50) and the related lateral shaft (60) are in an intermediate position between the front (12a) and rear (12b) of the handle (10), the size of the handle (10) being such as to enable the handle to be held comfortably in an operator's hand. The person skilled in the art can easily understand that the operator can hold the handle (10) tightly while moving the wheel (50).

According to yet another specific variant embodiment, the wheel (50) is mounted on a rotary shaft (51) positioned such as to ensure that the external circumferential edge of the wheel (50) projects outside the handle (10), passing through an opening (70) provided for this purpose in the housing.

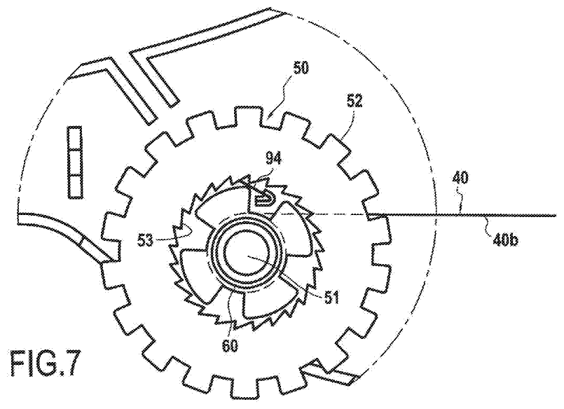

According to another specific variant embodiment, the external circumferential edge of the wheel (50) is notched and has notches (52) to facilitate the rotational movement imparted by the operator's finger or thumb when the wheel (50) is positioned on the upper portion of the handle (10), as shown. According to a variant, the wheel (50) may also be provided with a pawl (94) that cooperates with the teeth (53) provided, in a axial seat of the wheel (50), thereby preventing the untimely rotation of the wheel during insertion of the catheter (100), as clearly shown in FIG. 7.

According to yet another specific variant embodiment, a support and change-of-direction shaft (82) for the traction wire (40) coming from the front portion (12a) of the handle (10) through the loop (30) is provided inside the rear portion (12b) of the handle (10), which also helps to significantly lower the tractive force that needs to be exerted on the intermediate protective sheath (120) to pull said sheath inside the handle (10) and to release the stent (2).

According to another specific variant embodiment, the support and change-of-direction shaft (82) arranged at the rear of the handle includes a spacer (84) that is rigidly connected to the support shaft (82). The handle can also have a transverse reinforcement element (13) at the rear including a through-hole extending the central channel (111), as shown in is FIG. 6.

According to another specific variant embodiment, the handle (10) has a hollow reinforcement element (18) at the front (12a) into which the catheter (100) is inserted, thereby preventing the catheter device (100) including the stent (2) from kinking. According to the variant embodiment shown notably in the magnified view in FIG. 3, the reinforcement element (18) has a tapered front portion arranged outside the handle and an inverted-T-shaped rear portion in which the base of the “T” is seated in a transverse seat and held firmly in the closed position of the handle.

According to another specific variant embodiment, an element (90) for temporarily blocking any untimely movement of the tackle system (20) is provided.

According to a specific variant embodiment, the blocking element (90), such as a pin with a finger-hole (91) for pulling the pin, can be provided near to the loop (30), for example to be inserted beneath the loop (30), and for example has a cylindrical or tapered lug (92) used to lift and compress the loop in order to block the loop in position until the handle (10) is used. The blocking element (90) in this case advantageously has a second lug (93) that is inserted between two teeth (52) of the wheel (50), which also prevents the wheel from turning in an untimely fashion.

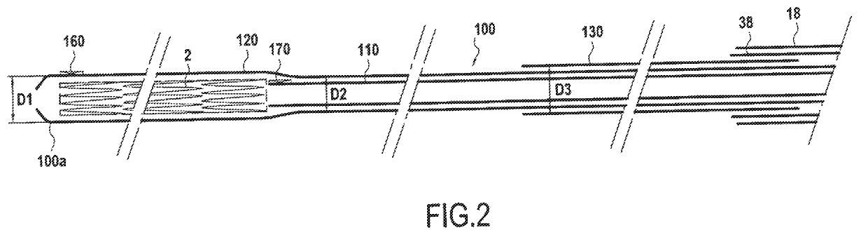

According to a second aspect, the invention also relates to a system for delivering a stent (2) positioned on a catheter (100), shown in greater detail in FIGS. 1 and 2, characterized in that it includes a handle (10) as defined above.

According to a specific variant embodiment, the system is characterized in that the catheter (100) has a distal end (100a) including the stent (2) to be delivered and a proximal end (100b), and is shaped to be inserted into a channel of the human body, in particular a blood vessel, vein, artery or peripheral application; including an intermediate tubular element or sheath (120) with a proximal end and a distal end; a stent (2) arranged in a constrained manner inside the distal end of the intermediate tubular element (120); an inner tubular element or sheath (110) with a proximal end and a distal end that is arranged inside the intermediate tubular element (120) and that is long enough that the distal end thereof is positioned at the rear of the stent and blocks the stent in position to prevent the stent from being retracted, as shown in FIG. 2; the proximal end of the inner tubular element (110) being rigidly connected to the handle (10) in a static position.

According to another specific variant embodiment of the invention, an outer sheath or tubular element (130) with a proximal end and a distal end is positioned outside the intermediate tubular element (120) and is shorter than the intermediate tubular element so that the distal end thereof is positioned at a predetermined distance upstream of the stent, as shown, and the proximal end of the outer tubular element (130) is rigidly connected to the handle (10) also in a static position.

According to another variant embodiment of the invention, the distal end of the intermediate tubular element (120) has a diameter D1 that is greater over at least the entire length along which the stent (2) is received to facilitate the insertion thereof, then has an inclined joint resulting in a lesser diameter D2 over the remainder of the intermediate tubular element, as shown in FIG. 2. This diameter D1 is substantially equal to is the diameter D3 of the outer tubular element (130) such as to be positioned against the distal end of the outer tubular element, which is static and forms a stop preventing retraction of the intermediate tubular element.

According to another variant, a tubular element (38) is provided inside the handle and can be transparent to be used as a movement guide for the movable element, such as the intermediate sheath (120) and the traction wire (40).

The person skilled in the art can easily understand that the intermediate tubular element (120) is designed to slide longitudinally between the inner tubular element (110) and the outer tubular element (130), which remain in a static position relative to the handle (10) to which said tubular elements are rigidly connected. The stent (2) remains in place because the distal end of the inner tubular element (110) remains static and prevents any retraction movement of the stent. The invention therefore enables the stent (2) to be delivered to a precise position.

The person skilled in the art will also understand that the outer tubular element (130) enables friction with the channel of the human body into which the catheter (100) is inserted to be reduced.

The benefit of the handle according to the invention over the existing “fixed point” system, which requires the use of an assistant, lies in the fact that the surgeon can use the system alone.

Indeed, the surgeon or operator can use one hand in the vicinity of the inserter (not shown here) to correct any undesired movement of the is protective intermediate tubular element or sheath (120) of the stent (2) in the channel such as a blood vessel of the patient that could result in the implant or stent (2) being deployed away from the site of the stenosis. The surgeon or operator can use the other hand to rotate the wheel (50) to release the stent (2) after the blocking element (90) forming a safety pin has been removed.

Conventionally and advantageously, position markers may be used, for example a distal marker (160) at the front end 100a of the catheter, in this case on the intermediate tubular element (120); a distal marker (170) at the front end of the inner tubular element (110) arranged behind the stent (2) to prevent the stent from being retracted. This ensures that the position of the stent (2) is perfectly indicated for the doctor.

The method for inserting the implant or stent (2) in the channel such as a blood vessel of the patient is described below:

an inserter is placed in the patient's artery: brachial or femoral approach,
a guide wire is inserted into the inserter and enters then passes the site where the implant or stent (2), which may advantageously have shape memory, is to be implanted.

The catheter (100) is then slid along the internal channel (111) thereof on the guide wire and inserted in the inserter before advancing to the delivery site of the stent (2), the advance position of the stent (2) being marked using the markers (160) and (170). During this placement, the catheter (100) is advantageously flushed out using a water/liquid contrast mixture in order to entirely eliminate any air from inside the catheter, including the inner and intermediate sheaths or elements (110, 120) is supporting and protecting the implant or stent (2), and the outer sheath or element (130) used for insertion of the catheter.

It can thus be understood that the distal end (100a) of the catheter (100) is engaged on the guide wire via the inserter in order to reach the implant site.

The operator then checks the placement of the stenosis by injecting a contrast product mixture in the arterial tree visible using radioscopy in order to position the implant or stent (2) at the site of the stenosis.

This implant or stent (2) is designed to be implanted in the peripheral arteries at the site of the stenosis (reduction in the diameter of the artery).

The preferred sites are the extremities of the limbs of the human body, in particular the legs, which may suffer from poor irrigation, behind the knees, etc.

The implant or stent (2) must be flexible to absorb any flexion, torsion, etc. created by natural limb movement.

The benefit of using a shape memory material, such as nitinol, to manufacture the implant or stent is the ability of the implant positioned in a superficial artery to return to the initial shape thereof after suffering an impact (table edge, etc.), unlike implants made of stainless steel or CrCo, which are not shape memory materials and would stay deformed. For example, a stent with a diameter of 7 mm is implanted in the stenosis of an artery in which the diameters upstream and downstream of the stenosis are approximately 6 mm.

The implant is then perfectly affixed to the wall and thus held in the arterial flow by the counterpressure thereof.

### Definition of Different Elements of the Handle According to the Invention

The handle (10) comprises two main matching parts, or a right-hand shell (11) shown in FIG. 1 and a left-hand shell (12) shown in FIGS. 3 to 8. The shape of the right- and left-hand shells is designed to enable the surgeon to use the handle easily and comfortably.

The size of the handle is relatively small to ensure that the device is not too large and the envelope too cumbersome, while enabling sufficient backward movement of the intermediate sheath or element, regardless of the stent in the range.

The reinforcement (18) is positioned at the front end of the handle (10) and is intended to prevent the sheaths from kinking following handling, which can be abrupt, by operators attempting to correctly position the stent in the stenosis.

The reinforcement is flexible, while being strong enough to perform the protective function.

The wheel (50):

The wire (40) attached to the wheel (50) can be wound up, in this case in particular about the lateral shaft (60) thereof when the surgeon decides to release the stent (2) by turning the wheel. The wheel (50) tightens the wire (40) passing through the loop (30) of the intermediate sheath or tubular element (120). Since the wire (40) is fastened at the other end (40a) thereof, the system perfectly forms a tackle. This structure halves the tractive force required to retract the intermediate sheath or tubular is element (120).

The diameters of the wheel (50) and of the lateral shaft (60) are optimized to facilitate the release of the stent (2): winding diameter of the wire (40) and total diameter of the wheel (50), where the notches (52) are formed.

The notches (52) are designed not to be too aggressive for the operator's gloves, while giving the operator sufficient grip to prevent slipping. A pawl system (94), for example a flexible blade such as a beater plate, prevents any inverse movement of the wheel (50).

The wedge 78: The role of the wedge is minimal, enabling the different components to be positioned inside the handle easily during assembly.

The base (80) is arranged to extend the inner tubular element 110, also known as “push tubing” or “stop tubing”, which is bonded to the inside of the base (80).

The base (80) and the inner tubular element (110) enable the guide wire to pass through the central channel (111) over the entire length of the delivery system including the catheter (100).

The wire (40) of the tackle (20) is also fastened to the inside of the base (80).

The transparent tube (38) extends inside the handle over almost the entire length thereof. The transparent tube has a notch (39) to enable passage of the blocking member. The wire (40) starts inside the base (80), where the wire is fastened by means of a knot 42 to the shell (12) towards the loop (30) of the intermediate gray sheath, then turns back towards the change-of-direction support (82) to be redirected towards the wheel (50) and the winder bearing (60) thereof.

The transparent tube (78) enables the guide, the inner tubular element is (110) and the intermediate element (120) to be kept straight in the handle when retracting the latter. It should be noted that the outer tubular element (130) is fastened to the front portion of the handle (10). Thus, the transparent tube (78) is bonded to the left-hand shell (12) to ensure same remains stiff when tensioned.

The tackle (20) operates in the space between the outside of the inner tubular element (110) or “push tubing” and the inside of the transparent sheath (78).

The loop (30) can comprise a wire with two legs bonded to the intermediate tubular element (120). The wire (40) is also used to move the intermediate tubular element (120) by pulling same through the loop (30) using the tackle system (20).

A change-of-direction support or metal pin (82) bonded in place on the left-hand shell (12) is used as a solid shaft to turn the wire coming from the base back towards the wheel. The metal pin (82) is also held by the right-hand shell (11) when the handle (10) is closed.

A spacer (84) bonded about the metal pin (82) is used to increase the radius of curvature thereof to ensure that the change of direction is efficient and continuous.

A no-return element (94), for example a beater plate, positioned beneath the axial portion of the wheel (50), is used to prevent the wheel from moving backwards when the wire (40) is tightened.

### Conclusion

The tackle system (20) according to the invention makes it possible to half the force exerted by the operator on the wheel (50) in order to retract or pull back the intermediate sheath or element (120). The loop (30) bonded to the base of the intermediate sheath or element (120) acts as “pulley” in which slides the retraction wire (40), which is connected at one end to the housing or in this case the base rigidly connected to the housing, forming a static point, and at the other end to the wheel (50), which is a moveable point.

This loop (30) could be replaced by a pulley, but the limited space resulting from the internal diameter of the transparent tube (78) means that the loop is the best means for performing this role. This tackle system (20) is entirely novel.

1-15. (canceled)

16. A handle for delivering a stent by retracting a tubular element or sheath for protecting the stent , including a tackle system used to pull the protective sheath over a predetermined retraction distance (D) to uncover the stent and enable the stent to be delivered and expanded, wherein the tackle system includes a loop that is rigidly connected on the upstream side to the protective sheath, through which passes a traction wire performing a tackle function, one end of which is fastened to the housing of the handle, and is therefore static, and the other end of which is rigidly connected to a control wheel that is mounted in rotation on the housing of the handle.

17. The handle as claimed in claim 16, wherein the handle has a front portion on the side of the stent and a rear portion used to insert a catheter on which the stent is mounted; in a starting position, the tackle system is positioned towards the front of the handle so that, when retracted by traction, the retraction distance (D) is substantially close to the length of the handle, this length being calculated in consideration of the length of the stent.

18. The handle as claimed in claim 16, wherein the wheel has a lateral shaft onto which the traction wire is wound.

19. The handle as claimed in claim 18, wherein the wheel and the related lateral shaft are in an intermediate position between the front and rear of the handle, the size of the rear portion of the handle being such as to enable the handle to be held comfortably in an operator's hand.

20. The handle as claimed in claim 16, wherein the wheel is mounted on a rotary shaft positioned such as to ensure that the external circumferential edge of the wheel projects outside the handle, passing through an opening provided for this purpose in the housing.

21. The handle as claimed in claim 20, wherein the external circumferential edge of the wheel is notched and has notches to facilitate the rotational movement imparted by the operator's finger or thumb.

22. The handle as claimed in claim 16, wherein a support and change-of-direction shaft for the traction wire coming from the front portion of the handle through the loop is provided inside the rear portion of the handle, which also helps to lower the tractive force that needs to be exerted on the protective sheath to pull said sheath inside the handle.

23. The handle as claimed in claim 22, wherein the support and change-of-direction shaft arranged at the rear of the handle includes a spacer that is rigidly connected to the support shaft.

24. The handle as claimed in claim 16, wherein the handle has a hollow reinforcement element at the front into which the catheter is inserted, thereby preventing the catheter device including the stent from kinking.

25. The handle as claimed in claim 16, wherein an element for temporarily blocking movement of the tackle system is provided.

26. The handle as claimed in claim 25, wherein the blocking element can be provided at the loop, for example by insertion beneath the loop, and can for example have a cylindrical or tapered lug designed to raise and compress the loop in order to block the loop in position until the handle is used, and advantageously also at the wheel to prevent rotation thereof.

27. A system for delivering a stent positioned on a catheter, comprising a handle as defined in claim 16.

28. The system as claimed in claim 27, wherein the catheter has a distal end including the stent to be delivered and a proximal end, and is shaped to be inserted into a channel of the human body, in particular a blood vessel, vein or peripheral artery; said catheter including an intermediate tubular element or sheath with a proximal end and a distal end; a stent arranged in a constrained manner inside the distal end of the intermediate tubular element; an inner tubular element or sheath with a proximal end and a distal end that is arranged inside the intermediate tubular element and that is long enough that the distal end thereof is positioned at the rear of the stent and blocks the stent in position to prevent the stent from being retracted; the proximal end of the inner tubular element is rigidly connected to the handle and is therefore static.

29. The system as claimed in claim 27, wherein the catheter comprises an outer tubular element that is provided to reduce friction with the channel of the human body, that is positioned outside the intermediate tubular element and that is shorter than the intermediate tubular element so that the distal end thereof is positioned at a predetermined distance upstream of the stent, and the proximal end of the outer tubular element is rigidly connected to the handle, also in a static position.

30. The system as claimed in claim 27, wherein the distal end of the intermediate tubular element has a diameter D1 that is greater over the entire length along which the stent is received, to facilitate the insertion thereof, then has an inclined joint resulting in a lesser diameter D2 over the remainder of the intermediate tubular element and this diameter D1 is advantageously substantially equal to the diameter D3 of the outer tubular element such as to be positioned against the distal end of the outer tubular element, which is static and forms a stop preventing retraction of the intermediate tubular element.
